(19) United States
(12) Patent Application Publication
(10) Pub. No.: US 2012/0125327 A1
(43) Pub. Date: May 24, 2012

(54) Nasal Passage Strip

(76) Inventor: Patricia Blair Huntington, Houston, TX (US)

(21) Appl. No.: 13/275,594

(22) Filed: Nov. 3, 2011

Related U.S. Application Data
(60) Provisional application No. 61/455,281, filed on Oct. 18, 2010.

(51) Int. Cl. A62B 23/06; A61M 16/10; A62B 23/00

(52) U.S. Cl. 128/203.12; 128/206.14; 128/206.18

(57) ABSTRACT

A nasal passage covering is provided to adhere to opposite sides of the lower nose. The covering further has a filtering material designed to cover the nostrils to filter air coming into the body.

### BACKGROUND OF THE INVENTION

1. Field of the Invention

The present invention is in the field of skin protection coverings, more particularly, in the field of nasal coverings.

2. Description of Related Art

Inhaling exposes the nose to contaminants that impact the health of a person. When entering an area with airborne allergens, contaminants, or germs, nose hairs are limited in their ability to fight unwanted or foreign particles and their ability to humidify the air is reduced. Unfiltered air enters the body through the nasal cavity.

Facial coverings or bandages may impact a person's ability to communicate, or may suggest personal health issues.

### SUMMARY OF THE INVENTION

The subject matter of this application may involve, in some cases, interrelated products, alternative solutions to a particular problem, and/or a plurality of different uses of a single system or article.

The present invention is a nasal passage covering with two separate adhesive strips of tape that adhere to opposite sides of the lower nose and are connected by a filtering material covering the nasal passages that allows for sufficient air intake.

Another aspect of the top side of the strip material is that it may have no adhesive coating.

Another aspect of the filter material is that it may be slightly pleated to offer additional filtration.

Another aspect of the filter material is that it may be coated for medical or aromatic purposes.

Uses of the nasal passage strips include protection for the nasal passages from invasion of unwanted or foreign particles such as allergens. Nasal passage strips allow air filtration and discourage the clogging of nose hairs which act as protective barriers. The disposable strips are designed with a filtering material centered under the nasal passages and anchored by adhesive strips on both sides of the lower nose.

The prior art provided coverings that cover additional facial features such as the mouth, require attachment to other areas of the head, such as the ears, or protect bone structure. Hence, the use of the other coverings may impair communications, imply personal health concerns, become bothersome to wear or provide excessive protection to additional facial features.

### BRIEF DESCRIPTION OF THE DRAWINGS

FIG. 1 provides a top assembled view of the nasal passage strip.

FIG. 2 provides a bottom assembled view of the nasal passage strip.

FIG. 3 provides a view of the transparent backing.

FIG. 4 provides a view of the bottom of the nasal passage strips showing the adhesive.

FIG. 5 provides a view of an embodiment of the filter material.

### BRIEF DESCRIPTION OF THE INVENTION

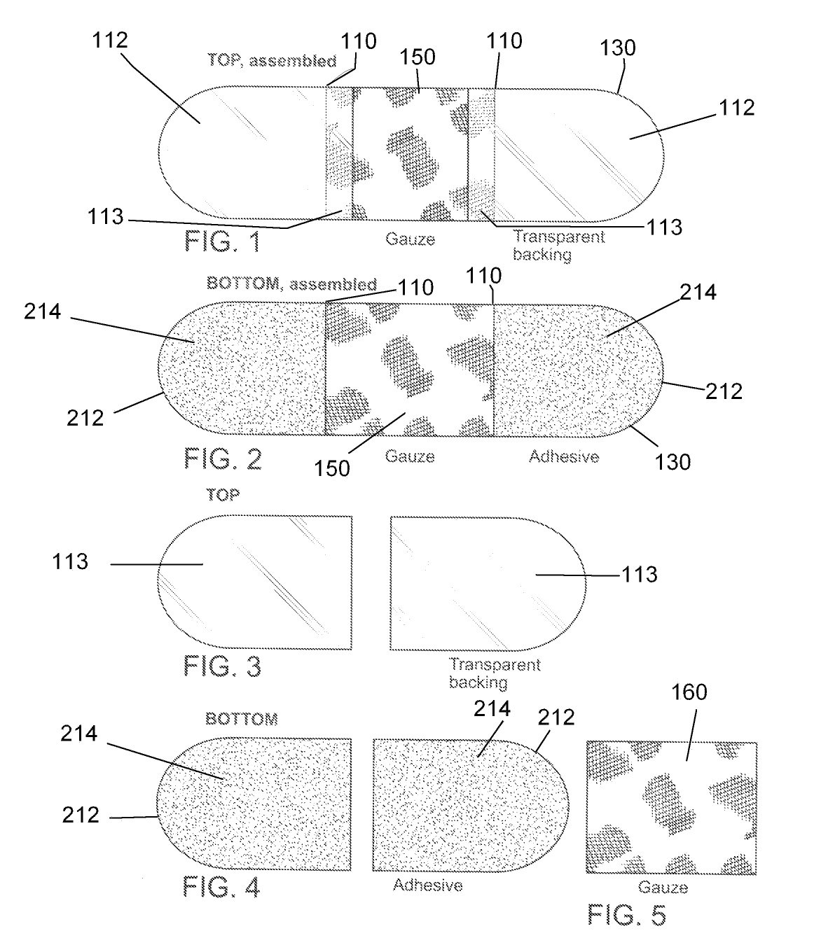

Referring to FIG. 1 there is shown a top side 112 of the nasal passage strip of an air impermeable or air permeable material such as a flexible plastic material or woven or non woven fabric or the like capable of receiving and retaining adhesive of the type typically used for adhesive bandages separated by a center section of filter material 150 that centers under the nasal passages (nostrils). Underside strip 212 with adhesive is denoted to indicate a difference as the top side 112 may have no adhesive. In this view, the top side 112 of two strips are shown, the strips being on opposite sides of the filter material 150. Further, FIG. 1 shows a transparent backing 113 which may be removably attached to the strip 112. The backing 113 may be positioned on the top side 112, or the bottom side 212 (see FIG. 2) of the strip. It should be understood that the backing 113 may be opaque or translucent without straying from the scope of this invention.

Referring to FIG. 1 there may be a curved or angular shape 130 on the opposite ends of the strips 112 to allow for fit and comfort. This same shape applies to the underside of the strip 212.

Referring to FIG. 1 there is a binding or stitching 110 that attaches the filter material to the sections of strip.

Referring to FIG. 1 there is a section of air permeable filter material 150 that invites air passage while limiting the intake of unwanted or foreign objects.

Referring to FIG. 2 there is shown a bottom side 212 of the nasal passage strip with an adhesive coating 214 sufficient to adhere the strip to the external sides of the nose. Wherein the strips 112 are separated by a center section of filter material 150 such as gauze that is sized to center under the nasal passages. Further, there may be a curved or angular shape 130 on the opposite ends of the strips 112 to allow for fit and comfort. A quantity of adhesive 214 is coated on the surface of the strip 212.

FIG. 3 shows a detail view of an embodiment of the transparent backing 113. The transparent backing 113 has substantially the same shape as the strip (as shown in FIG. 1).

FIG. 4 shows a detail view of an embodiment of the bottom of the strips 212. An adhesive 214 is coated on the surface of the strip 212.

Referring to FIG. 5 there is an alternative section of filter material 160 with pleating that may increase the collection of unwanted or foreign particles. This material may be used as an alternative to the less compacted filtering material. FIG. 5 shows a replacement item for filter material 150.

In a further embodiment, two additional thin strips may be positioned on the two sides of the filter material that do not have strips attached.

In another embodiment, a single strip may be employed. In a further embodiment, the strip may form an aperture at its center. The aperture may be large enough to cover both nostrils of a user, and the strip may be large enough to be attached to each outer side of a user's nose. A filter material may be attached to the strip and may cover the aperture formed by the body. An adhesive covering on a surface of the strip allows the strip to be attached to opposing outer sides of a user's.

While several variations of the present invention have been illustrated by way of example in preferred or particular embodiments, it is apparent that further embodiments could be developed within the spirit and scope of the present invention, or the inventive concept thereof. However, it is to be expressly understood that such modifications and adaptations are within the spirit and scope of the present invention, and are inclusive, but not limited to the following appended claims as set forth.

What is claimed is:

1. A nasal passage covering comprising:
a substantially air-impermeable strip;
a quantity of adhesive coated on a bottom side of the strip;
a filter material, a first side of the filter material attached to a side of the strip, the filter material being configured to be permeable to air and to limit passage of a foreign object; and
wherein the strip and filter material are attached such that they are sized and configured to be attached to a side of a nose of a user and sized and configured to position the filter material flush against a nostril of the user.

2. The nasal passage covering of claim 1 further comprising a backing material removably attached to the bottom side of the strip.

3. The nasal passage covering of claim 1 further comprising a binding to attach the filter material to the side of the strip.

4. The nasal passage covering of claim 1 wherein the filter material is coated with a substance capable of inhalation.

5. The nasal passage covering of claim 4 wherein the substance capable of inhalation is a medicinal substance.

6. The nasal passage covering of claim 1 wherein the filter material is configured and sized to fit over both nostrils of a user.

7. The nasal passage covering of claim 1 further comprising a second strip, a side of the second strip attached to a second side of the filter material.

8. The nasal passage covering of claim 7 wherein the second strip is sized and configured to be attached to a side of a nose of a user, and further comprising a quantity of adhesive coated on a bottom side of the second strip.

9. The nasal passage covering of claim 1 wherein the filter material is a pleated material.

10. A nasal passage covering comprising:
a strip, the strip sized and configured to be attached to two sides of a nose of a user, the strip forming an aperture sized to allow air passage through a nostril of the user;
a quantity of adhesive coated on a bottom side of the strip; and
a filter material attached to the strip and covering the aperture formed by the strip.

11. The nasal passage covering of claim 10 further comprising a backing material removable attached to the bottom side of the strip.

12. The nasal passage covering of claim 10 further comprising a binding to attach the filter material to the side of the strip.

13. The nasal passage covering of claim 10 wherein the filter material is coated with a substance capable of inhalation.

14. The nasal passage covering of claim 13 wherein the substance capable of inhalation is a medicinal substance.

15. The nasal passage covering of claim 10 wherein the filter material is configured and sized to fit over both nostrils of a user.

16. The nasal passage covering of claim 10 wherein the filter material is a pleated material.

17. A method of limiting intake of airborne contaminants comprising the steps of:
positioning a filter of a nasal passage covering tightly over a nostril of a user; and
attaching a strip of the nasal passage covering to an outer surface of a nose of a user.

18. The method of limiting intake of airborne contaminants of claim 17 further comprising the step of utilizing an adhesive coated on a bottom side of the strip to attach the strip of the nasal passage covering to the outer surface of the nose of the user.

19. The method of limiting intake of airborne contaminants of claim 18 further comprising the step of positioning the filter over a second nostril of the user.

20. The method of limiting intake of airborne contaminants of claim 19 further comprising the step of removing a backing that is removably attached to the bottom of the strip.
